(19) United States
(12) Patent Application Publication
(10) Pub. No.: US 2017/0065526 A1
(43) Pub. Date: Mar. 9, 2017

(54) SOLID DOSAGE FORM OF RIVAROXABAN AND METHODS FOR MAKING THE SAME

(71) Applicants: Shaoping Wang, Nanjing (CN); Xin Liu, Nanjing (CN); Meng Wang, Nanjing (CN); Weining Yang, Los Angeles, CA (US); Kwok Chow, Missisauga (CA)

(72) Inventors: Shaoping Wang, Nanjing (CN); Xin Liu, Nanjing (CN); Meng Wang, Nanjing (CN); Weining Yang, Los Angeles, CA (US); Kwok Chow, Missisauga (CA)

(21) Appl. No.: 15/259,039

(22) Filed: Sep. 7, 2016

(30) Foreign Application Priority Data
Sep. 8, 2015 (CN) 201510567761X

(51) Int. Cl. A61K 9/20; A61K 31/5377; A61K 9/28; A61K 9/00

(52) U.S. Cl. CPC A61K 9/2095; A61K 9/0053; A61K 31/5377; A61K 9/28; B01J 2/04

(57) ABSTRACT

The present invention discloses a pharmaceutical composition that includes rivaroxaban and one or more excipient in a solid dosage form and methods for making the same. Methods for making compositions of the present invention includes powderizing rivaroxaban by centrifugal wet granulation to form compositions suitable for solid oral dosage form. Pharmaceutical dosage forms produced by methods of the present invention are more homogenous, smoother, and have better rheological properties, better compressibility, and much easier to make. They are much lower in cost and also easier to produce at industrial scales.

### CROSS-REFERENCE TO RELATED APPLICATIONS

This application claims the benefit of Chinese Patent Application Number 201510567761X, file on Sep. 8, 2015. The above application(s) is hereby incorporated herein by reference.

### FIELD OF THE INVENTION

The present invention pertains to the field of pharmaceutical formulation. More particularly, the present invention relates to the formulation of a solid dosage form of rivaroxaban as a highly selective inhibitor of factor Xa and method for manufacturing the same.

### BACKGROUND OF THE INVENTION

Normal process of blood coagulation serves the purpose of maintaining the structural integrity of blood vessels. However, abnormal or pathological coagulation can occur in many clinical conditions. Abnormal blood coagulation include deep venousthrombosis (DVT) and its main complication—pulmonary embolism (PE), as well as stroke, and other systemic presentations caused by heart embolism. The goal of anticoagulating medication is to prevent the formation or expansion of pathological blood coagulation in patients so as to reduce the risk of pathological thrombosis.

In addition to the three commonly used anticoagulants (i.e. heparin, low molecular weight heparin, and dihydroxycoumarin), there are now several newer generation of anticoagulants garnering the attention of drug developers. Two of these new anticoagulants deserve particular mention. The first type of new anticoagulants are direct thrombin inhibitors and their formulations such as Bivalirudin. The second type of new anticoagulants are factor Xa inhibitors and their formulations, including both direct and indirect factor Xa inhibitors. Examples of this type of anticoagulants include indirect factor Xa inhibitors such as Fondaparinux and direct factor Xa inhibitors such as rivaroxaban. Among direct factor Xa inhibitors, rivaroxaban has completed full scale clinical trials for reducing the risk of blood clots after total hip joint or knee joint replacement surgeries. It had received marketing approval in EU and Canada On September 15 and October 1 of 2008, respectively. On July 1 and Nov. 4, 2011, it further received FDA approval for clinical use as prophylaxis for DVT and PE during hip or knee surgery.

The systematic IUPAC name of rivaroxaban is (S)-5-chloro-N-{[2-oxo-3-[4-(3-oxomorpholin-4-yl)phenyl]oxazolidin-5-yl]methyl}thiophene-2-carboxamide. Its chemical formula is C₁₉H₁₈ClN₃O₅S and has a molecular weight of 435.89. The structural formula for rivaroxaban is as shown below:

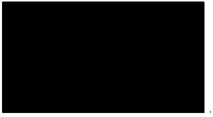

Physically, rivaroxaban exists as white to light yellow power and has relatively poor solubility (˜7 mg/L). As such, a method is needed to formulate rivaroxaban into an oral dosage form that can meet the requirements of dosage concentration and dissolution, amongst other dosage form requirements.

CN 1886120A discloses a method for preparing an oral dosage form composition comprising rivaroxaban. The composition includes a hydrophilic form of rivaroxaban to form the oral dosage form via the technique of fluid bed granulation. However, granules obtained from fluid bed granulation has large polydispersity, do not have sufficiently uniform roundness and a wide spread of diameter dispersion. Such composition has more fine powders, show poor rheology and compressibility, and do not perform ideally in real-world manufacturing process.

Therefore, there still exists a need for a more effective method of formulating rivaroxaban solid oral dosage forms.

### SUMMARY OF THE INVENTION

In light of the above shortcomings of the prior art, inventors of the present invention had set out to devise a better method for producing rivaroxaban particles more suited for formulating solid oral dosage forms. Through extensive trial-and-error experimentation, the inventors of the present invention unexpected discovered that centrifugal wet granulation-based methods of granulizing rivaroxaban can produce rivaroxaban particles with better physical properties suited for formulating a solid dosage form, including more uniformly round shapes, superior rheology properties, better compressibility and solubility.

Accordingly, in one aspect, the present invention provides a method for preparing a granulated rivaroxaban suitable for formulating a solid oral dosage form.

Methods in accordance with the present invention may be performed by centrifugal wet granulators generally known in the art. In general, such granulators will include a rotary base and a ventilation/heating mechanism. During operation, the rotary base typically will have a rotation speed range of between 0 to about 1000 rpm, preferably from about 100 to about 500 rpm, and more preferably from about 150 to about 300 rpm.

In a preferred embodiment, the rotation speed of the rotary base is set between about 100 to about 500 rpm, incoming air temperature is set at about 50° C. to about 80° C., ventilation frequency set at about 10.0 to about 20.0 Hz, air venting frequency set at about 20.0 to about 30.0 Hz, atomization pressure is set at about 0.5 to about 1.5 bar, the rotation speed of peristaltic pump is set at about 5 to about 20 rpm. The granulator is pre-heated to the temperature of the feed stock up to about 30° C. The above described centrifugal granulator further includes one or more atomizing nozzles. A rivaroxaban suspension is injected and atomized at a constant rate until granulation is completed.

In another aspect, the present invention provides pharmaceutical preparation of rivaroxaban composition produced in accordance with the above described method. In some prefer embodiments, about 90% of the rivaroxaban granules have diameters less than 50 micrometer, preferably less than 25 micrometer, and more preferably less than 15 micrometer.

In some other embodiments, the preparation includes at least one excipient. In some other embodiments, the at least one excipient may be a disintegrating agent. In still some other embodiments, the at least one excipient may be a thinning agent.

In still some other embodiments, the preparation includes an excipient comprising the combination of at least one disintegrating agent, and at least one thinning agent. In other embodiments, the preparation includes an excipient comprising the combination of at least one thinning agent and one adhesive agent, or the combination of at least one thinning agent, at least one disintegrating agent, and at least one adhesive agent.

In yet another embodiment, the preparation includes a crystalline form of rivaroxaban.

In another aspect, the present invention provides a pharmaceutical composition in a solid oral dosage form that includes rivaroxaban preparation as described above.

Pharmaceutical composition in accordance with the present invention may include one or more ingredients. The ingredients can be the same type or different types of excipients. In some embodiments, the excipient includes at least one disintegrating agent or may be a combination of a plurality of disintegrating agents; it may also include at least one thinning agent, or a combination of one or more adhesive agents. It may also include at least one thinning agent and at least one adhesive agent; at least one thinning agent and at least one disintegrating agent; or any combinations thereof.

Disintegrating agents may be any such agents generally known in the art and are not particularly limited. Exemplary disintegrating agents may include Polyvinylpyrrolidone, Crosslinked polyvinylpyrrolidone, Carboxymethylcellulose, Low-substituted hydroxypropyl methyl cellulose, Cross-linked carboxymethyl cellulose, Methylcellulose, alga, Sodium starch glycolate, starch, formaldehyde, casein, or any combination thereof.

Exemplary thinning agent may include maltose, Maltodextrin, lactose, fructose, dextrin, Microcrystalline cellulose, Pre-gelatinized starch, Sorbitol, sucrose, Silicate microcrystalline, cellulose, Powdered cellulose, Dextrates, Copovidone, mannitol, glucose, calcium phosphate, and any combinations thereof, but are not limited thereto.

Exemplary adhesive agent may include Gum arabic, dextrin, starch, polyvinylpyrrolidone, carboxymethylcellulose, guar gum, hydroxypropyl methylcellulose, methylcellulose, polymethacrylates, maltodextrin, hydroxyethyl cellulose, and any combination thereof, but not limited thereto.

Methods of preparing compositions of the present invention utilizes centrifugal granulation. Apparatus or granulators suitable for use generally include a rotary base, and an air ventilation and heating mechanism. During operation, the rotary base is preferably rotating at about 0 to about 1000 rpm, more preferably 100-500 rpm, even more preferably at 150-300 rpm. It may also include one or more atomizing nozzle.

Pharmaceutical composition of the present invention will include 5-30 mg of rivaroxaban, preferably 10-20 mg rivaroxaban. The rivaroxaban preferably has crystalline form.

The pharmaceutical composition of the present invention may also be coated with an enteric coating. Any enteric coating known in the art may be suitably used. Exemplary enteric coating may include Hydroxypropyl methyl cellulose, hydroxyethyl cellulose, polyvinyl alcohol based coating, but not limited thereto.

If the pharmaceutical composition is in the form of a tablet, it may include 0.1-0.7% of thinning agent and/or 0.1-50% of disintegrating agent. All percentages are by weight.

Prior art method of fluidized bed granulation utilizes air flow that rapidly blows from below to pass through the granules. Granules formed by prior art methods suffer from the shortcoming that they are generally quite porous with large surface area.

| | Different | |
|---|---|---|
| | granule structure | Different physical property |
| Centrifugal | Powder settles | Smooth surface [symbol] non-porous [symbol] |
| | on surface of | high strength |
| | granular nucleus | |
| Fluidized | Granular nucleus | porous [symbol] soft granules |
| bed | bond with each | |
| | other to form | |
| | bridge-like | |
| | structure | |

Embodiments of the present invention generally have the following advantages:

(1) granules formed by methods of the present invention are more spherical, has superior rheological properties and do not aggregate.

(2) granules formed by methods of the present invention has high compressibility, and disintegrates rapidly.

(3) methods of the present invention results in high yield, low cost.

(4) centrifugal granulators have numerous adjustable parameters, allow tuning of the granules made therefrom.

(5) centrifugal granulators are widely available, have small physical footprint and relatively inexpensive to operate.

Other aspects and advantages of the invention will be apparent from the following description and the appended claims.

### BRIEF DESCRIPTION OF THE DRAWINGS

FIG. 1 shows exemplary dissolution curves of pharmaceutical compositions in accordance with the different embodiments of the present invention. The dissolution test is performed in a solution containing 0.5% SLS (sodium lauryl sulfate) and pH 4.5 acetic acid-sodium acetate buffer.

FIG. 2 shows exemplary dissolution curves of pharmaceutical compositions in accordance with the different embodiments of the present invention in water.

FIG. 3 shows exemplary dissolution curves of pharmaceutical compositions in accordance with the different embodiments of the present invention in 0.1 mol/L of hydrochloric acid solution.

FIG. 4 shows exemplary dissolution curves of pharmaceutical compositions in accordance with the different embodiments of the present invention in a pH .5 acetic acid-sodium acetate buffer solution.

FIG. 5 shows exemplary dissolution curves of pharmaceutical compositions in accordance with the different embodiments of the present invention in a pH 6.8 phosphate buffer.

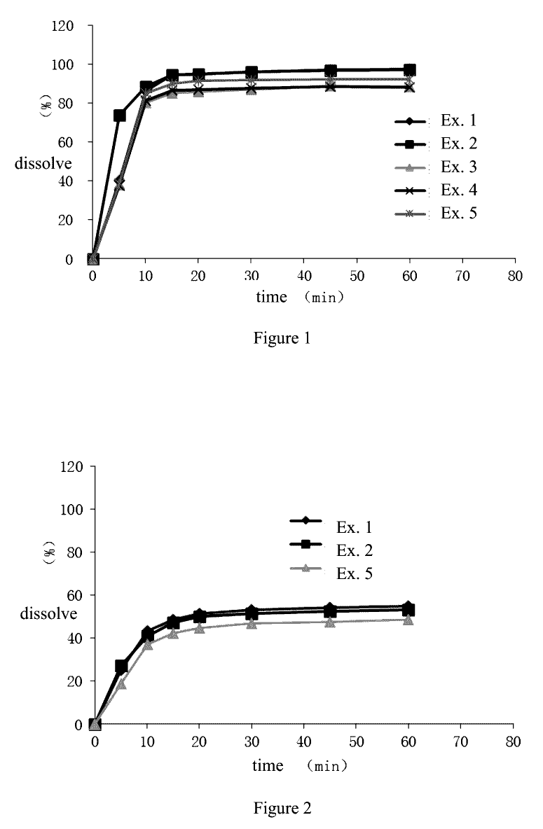

### DETAILED DESCRIPTION

The present invention will now be described in detail by referring to specific embodiments as illustrated in the accompanying figures.

To further illustrate the present invention, the following specific examples are provided

### EXAMPLES

### Example 1

### Manufacturing of 20.0 mg Rivaroxaban Tablets Using Centrifugal Granulation

1.1. Tablet Ingredients (Mg/Tablet)

| | |
|---|---|
| Rivaroxaban (micronized) | 20.0 mg |
| Microcrystalline cellulose | 36.0 mg |
| Lactose | 23.4 mg |
| Cross-linked sodium carboxymethyl cellulose | 3.0 mg |
| Hydroxypropylmethylcellulose, 5 cp | 1.5 mg |
| Sodium dodecyl sulfate | 0.5 mg |
| Magnesium stearate | 0.6 mg |
| enteric-coating material | 2.5 mg |

1.2 Preparation:

Dissolve Hydroxypropylmethylcellulose (5 cp) and Sodium dodecyl sulfate in water. Stir the mixture while adding micronized rivaroxaban. After all rivaroxaban is added, thoroughly mix to form a suspension. Introduce Microcrystalline cellulose, lactose, Cross-linked sodium carboxymethyl cellulose into centrifugal granulator. Set rotary speed to between 100-500 rpm. Air temperature to between 50˜80° C., ventilation frequency between 10.0˜20.0 Hz, air venting frequency to between 20.0˜30.0 Hz, atomizing pressure to between 0.5˜1.5 bar, peristaltic pump rotation rate to between 5˜20 rpm. Turn on the granulator and set initial granulator pre-heating temperature to 30° C., then initiate atomization and peristaltic pump to atomize the rivaroxaban suspension at a constant speed until all suspension is atomized. Turn off the atomizer and peristaltic pump. Allow the preparation material to dry until reaching 40° C. Then, discharge the preparation, pelletize and add Magnesium stearate to the mixture and thoroughly mix. Compress the resulting mixture to form 6 mm diameter tablets with breaking strength between 50-100N. Apply enteric coating to form the final tablet.

### Example 2

### Manufacturing of 15.0 mg Rivaroxaban Tablets Using Centrifugal Granulation

2.1 Tablet Ingredients (Mg/Tablet)

| | |
|---|---|
| Rivaroxaban (micronized) | 15.0 mg |
| Microcrystalline cellulose | 38.5 mg |
| Lactose | 25.9 mg |
| Cross-linked sodium carboxymethyl cellulose | 3.0 mg |
| Hydroxypropylmethylcellulose, 5 cp | 1.5 mg |
| Sodium dodecyl sulfate | 0.5 mg |
| Magnesium stearate | 0.6 mg |
| enteric-coating material | 2.5 mg |

2.2 Preparation:

Dissolve Hydroxypropylmethylcellulose (5 cp) and Sodium dodecyl sulfate in water. Stir the mixture while adding micronized rivaroxaban. After all rivaroxaban is added, thoroughly mix to form a suspension. Introduce microcrystalline cellulose, lactose, Cross-linked sodium carboxymethyl cellulose into centrifugal granulator. Set rotary speed to between 100-500 rpm. Air temperature to between 50˜80° C., ventilation frequency between 10.0˜20.0 Hz, air venting frequency to between 20.0˜30.0 Hz, atomizing pressure to between 0.5˜1.5 bar, peristaltic pump rotation rate to between 5˜20 rpm. Turn on the granulator and set initial granulator pre-heating temperature to 30° C. Then, initiate atomization and peristaltic pump to atomize the rivaroxaban suspension at a constant speed until all suspension is atomized. Turn off the atomizer and peristaltic pump. Allow the preparation material to dry until reaching 45° C. Then, discharge the mixture, pelletize and add Magnesium stearate to the mixture and thoroughly mix. Compress the resulting mixture to form 6 mm diameter tablets with breaking strength between 50-100N. Apply enteric coating to form the final tablet.

### Example 3

### Manufacturing 20.0 mg of Rivaroxaban Tablet Using High Shear Wet Granulation

3.1 Tablet Ingredients (Mg/Tablet)

| | |
|---|---|
| Rivaroxaban (micronized) | 20.0 mg |
| Microcrystalline cellulose | 36.0 mg |
| Lactose | 23.4 mg |
| Cross-linked sodium carboxymethyl cellulose | 3.0 mg |
| Hydroxypropylmethylcellulose, 5 cp | 1.5 mg |
| Sodium dodecyl sulfate | 0.5 mg |
| Magnesium stearate | 0.6 mg |
| enteric-coating material | 2.5 mg |

3.2 Preparation:

Dissolve Hydroxypropylmethylcellulose (5 cp) and Sodium dodecyl sulfate in water. Stir the mixture while adding micronized rivaroxaban. After all rivaroxaban is added, thoroughly mix to form a suspension. Introduce Microcrystalline cellulose, lactose, Cross-linked sodium carboxymethyl cellulose into high shear wet granulator. Set stirring rod speed to 250 rpm, shearing knife speed to 600 rpm, atomization pressure to 1.0 bar, peristaltic pump rotation speed to 20 rpm. Turn on the high shear wet granulator for 5 min, then, turn on atomizer and peristaltic pump to inject the suspension at a constant rate until all suspension is used up. Turn off atomizer and peristaltic pump. Quickly transfer the wet pellets to fluidized bed for drying. Set the air temperature of the fluidize bed to 65° C., ventilation frequency to 25.0 Hz, and begin drying until reaching 42° C. Then, take out the pellet, add magnesium stearate to the mixture and thoroughly mix. Compress the resulting mixture to form 6 mm diameter tablets with breaking strength between 50-100N. Apply enteric coating to form the final tablet.

### Example 4

### Manufacturing of 20.0 mg Rivaroxaban Tablets Using Direct Compression

4.1 Tablet Ingredients (Mg/Tablet)

| | |
|---|---|
| Rivaroxaban (micronized) | 20.0 mg |
| Microcrystalline cellulose | 36.0 mg |
| Lactose | 23.4 mg |
| Cross-linked sodium carboxymethyl cellulose | 3.0 mg |
| Hydroxypropylmethylcellulose, 5 cp | 1.5 mg |
| Sodium dodecyl sulfate | 0.5 mg |
| Magnesium stearate | 0.6 mg |
| enteric-coating material | 2.5 mg |

4.2 Preparation:

Thoroughly mix Sodium dodecyl sulfate with rivaroxaban powder. Then add microcrystalline cellulose, lactose, hydroxypropylmethylcellulose (5 cp) and cross-linked sodium carboxymethyl cellulose. Mix thoroughly and then finally add Magnesium stearate and mix thoroughly. Then, compress the resulting mixture to form 6 mm diameter tablets with breaking strength between 50-50N. Apply enteric coating to form the final tablet.

### Example 5

### Manufacturing 20.0 mg Rivaroxaban Tablets With Fluidized Bed

5.1 Tablet Ingredients (Mg/Tablet)

| | |
|---|---|
| Rivaroxaban (micronized) | 20.0 mg |
| Microcrystalline cellulose | 36.0 mg |
| Lactose | 23.4 mg |
| Cross-linked sodium carboxymethyl cellulose | 3.0 mg |
| Hydroxypropylmethylcellulose, 5 cp | 1.5 mg |
| Sodium dodecyl sulfate | 0.5 mg |
| Magnesium stearate | 0.6 mg |
| enteric-coating material | 2.5 mg |

5.2 Preparation:

Dissolve Hydroxypropylmethylcellulose (5 cp) and Sodium dodecyl sulfate in water. Stir the mixture while adding micronized rivaroxaban. After all rivaroxaban is added, thoroughly mix to form a suspension. Introduce Microcrystalline cellulose, lactose, Cross-linked sodium carboxymethyl cellulose into fluidized bed granulator. Set air temperature to 65° C., ventilation frequency to 20.0 Hz, atomizing pressure to 0.5 bar, peristaltic pump speed to 5 rpm. Set pre-heating temperature to 35° C., then turn on peristaltic pump to begin injecting and atomizing then suspension at a constant rate until all suspension is used up. Turn off the atomizer and peristaltic pump. Allow mixture to continue drying until reaching 50° C. Remove the material from the granulator, add magnesium stearate to the mixture and thoroughly mix. Compress the resulting mixture to form 6mm diameter tablets with breaking strength between 50-100N. Apply enteric coating to form the final tablet.

### Example 6

### Comparison of Rivaroxaban Granules Made by Different Preparation Methods

6.1 Comparing Shape, Diameter, Flowability, and Compressibility

Because preparation of Example 1 and 2 have very similar properties, they will be averaged and compared to the preparations of Examples 3 and 5.

| | | | flowability | |
|---|---|---|---|---|
| | | | ( repose | Compressibility |
| Prep Method | Shape | Diameter | angle ) | ( Carr's index ) |
| Fluidized bed | Near spherical [symbol] not | Wide spread [symbol] | 36.5° | 22.1 |
| ( Ex. 5 ) | round enough [symbol] | more fine powder | | |
| | porous | | | |
| High shear | Near spherical [symbol] not | Wide spread [symbol] | 36.9° | 20.8 |
| ( Ex. 3 ) | round enough [symbol] | few fine powder | | |
| | non-porous | | | |
| Centrifugal | spherical [symbol] non- | Narrow spread [symbol] | 33.1° | 18.2 |
| ( Ex. 1 or 2 ) | porous | few fine | | |
| | | powder [symbol] uniform | | |

6.2 Comparison of Yield

| Prep method | Yield |
|---|---|
| Fluidized bed ( Ex. 5 ) | 88.2% |
| High shear ( Ex. 3 ) | 86.2% |
| Centrifugal ( Ex.1 or 2 ) | 94.5% |

### Example 7

### Comparison of Tablets Made by Different Methods

7.1 Breaking Strength (Huanghai Medicine & Drug, YPD-300C Tablet Hardness Tester)
Example 1 tablet: 62N
Example 2 table: 63N
Example 3 tablet: 60N
Example 4 tablet: 43N
Example 5 tablet: 58N

7.2 Disintegration Time (Pharmacopoeia of the People's Republic of China 2010, Part 2, Appendix X A Disintegration Testing Method).

Example 1 tablet: 5.1 min

Example 2 tablet: 5.2 min

Example 3 tablet: 6.3 min

Example 4 tablet: 2.0 min

Example 5 tablet: 5.5 min

7.3 In Vitro Dissolution Test

Dissolution method (Pharmacopoeia of the People's Republic of China 2010, Part 2, Appendix X C Dissolution Method 2)

Stirring rod speed: 75 rpm

Dissolution medium:
1) 0.4% Sodium dodecyl sulfate, pH 4.5 acetic acid-sodium acetate buffer
2) Water
3) 0.1 mol/L hydrochloric acid
4) pH 4.5 acetic acid-sodium acetate buffer
5) pH 6.8 phosphate buffer

Dissolution medium temperature: 37° C.±0.5° C.

Detection method: HPLC (Pharmacopoeia of the People's Republic of China 2010, Part 2, Appendix IV D)

Detection wavelength: 250 nm

Calculation method: external standard

Reference dissolved solution: weigh accurately amount of rivaroxaban, add 50% acetonitrile, then dilute with dissolution medium to 25 μg/ml solution.

Use 6 tablets from each Example. Take sample at 5 min, 10 min, 15 min, 20 min, 30 min, 45 min, 60 min intervals and refill medium accordingly. Filter the sample and determine concentration. The following table shows the result:

| | 5 min | 10 min | 15 min | 20 min | 30 min | 45 min | 60 min |
|---|---|---|---|---|---|---|---|
| Ex. 1 | 40.2% | 86.5% | 94.8% | 95.0% | 95.2% | 97.3% | 97.7% |
| Ex. 2 | 73.9% | 88.6% | 94.5% | 94.9% | 95.3% | 96.9% | 97.1% |
| Ex. 3 | 38.2% | 80.2% | 85.1% | 86.0% | 86.9% | 89.1% | 88.7% |
| Ex. 4 | 37.6% | 81.4% | 86.9% | 87.1% | 87.3% | 88.6% | 88.4% |
| Ex. 5 | 38.8% | 85.2% | 90.1% | 91.5% | 91.2% | 92.3% | 92.5% |

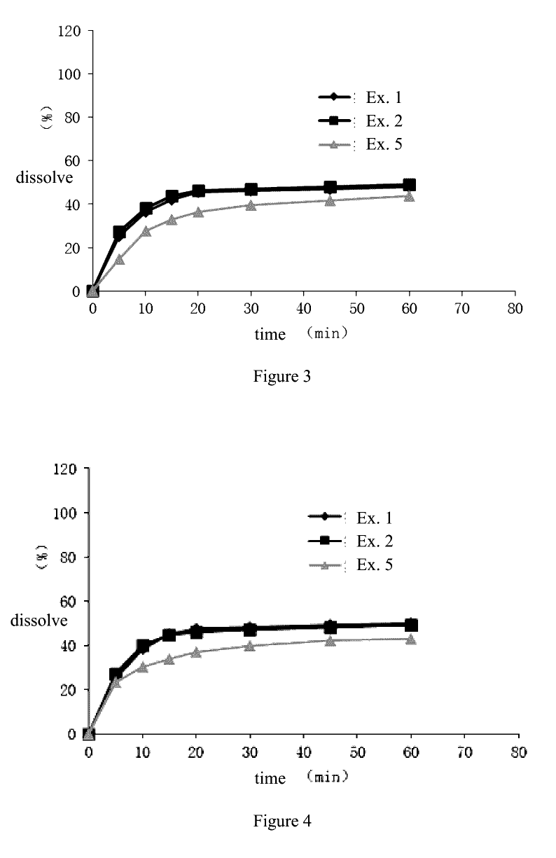

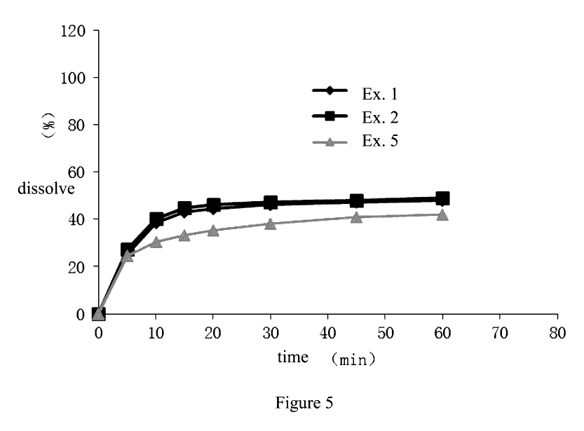

In addition, dissolutions of Example 1, 2 and 5 tablets in water, 0.1 mol/L hydrochloric acid, pH 4.5 acetic acid-sodium acetate buffer, and pH 6.8 phosphate buffer were also determined. Results are shown in FIGS. 2-5.

The above results show that solid dosage form of rivaroxaban obtained by centrifugal wet granulation method as disclosed in the present invention exhibits superior properties as a pharmaceutical formulation, particularly in simulated enteric environment, and is suitable for oral dosage form.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A method for preparing a composition comprising rivaroxaban and one or more excipients, said comprising:
forming an oral dosage form comprising micronized rivaroxaban using centrifugal wet granulation;
wherein said dosage form is suitable as a solid oral pharmaceutical composition.

2. The method of claim 1, wherein said method is performed by a centrifugal granulator.

3. The method of claim 2, wherein said granulator comprises a rotary base, an air ventilation and heating device, and said rotary base rotates at a rate of about 0 to about 1000 rpm during operation.

4. The method of claim 2, wherein said granulator further comprises one or more atomizing nozzle.

5. The method of claim 1, wherein said 90% of resulting rivaroxaban has a diameter less than 50 micrometer.

6. The method of claim 1, wherein the excipient comprise at least one disintegrating agent.

7. The method of claim 1, wherein the excipient comprises at least one thinning agent.

8. The method of claim 1, wherein the excipient comprises the combination of:
at least one thinning agent, and at least one disintegration agent;
at least one thinning agent, and at least one adhesive agent; or
at least one thinning agent, at least one disintegrating agent, or at least one adhesive agent.

9. The method of claim 1, wherein said solid oral pharmaceutical composition comprises crystalline form of rivaroxaban.

10. A solid oral pharmaceutical obtained according to the method of claim 1.
